(19) United States
(12) Patent Application Publication
(10) Pub. No.: US 2026/0324984 A1
(43) Pub. Date: Oct. 22, 2026

(54) AUTOMATED DETECTION AND TRACKING OF CORTICAL BLOOD VESSEL TEMPERATURE DURING NEUROSURGERY

(71) Applicant: Northwestern University, Evanston, IL (US)

(72) Inventors: Michael Alexander Iorga, Evanston, IL (US); Todd B. Parrish, Evanston, IL (US); Matthew Christopher Tate, Evanston, IL (US); Nils Jakob Schneider, Evanston, IL (US)

(21) Appl. No.: 19/105,561

(22) PCT Filed: Aug. 23, 2023
(86) PCT No.: PCT/US2023/072714

§ 371 Date: Feb. 21, 2025

Related U.S. Application Data
(60) Provisional application No. 63/400,591, filed on Aug. 24, 2022.

(51) Int. Cl. A61B 5/01; A61B 5/00; A61B 5/02; G06T 7/00; G06T 7/11; G06T 7/246; G06T 7/33; G06V 10/26; G06V 10/34; G06V 10/44; G06V 10/762; G16H 15/00

(52) U.S. Cl. CPC A61B 5/015; A61B 5/0077; A61B 5/02007; A61B 5/4064; G06T 7/0014; G06T 7/11; G06T 7/248; G06T 7/337; G06V 10/26; G06V 10/34; G06V 10/44; G06V 10/7625; G16H 15/00; G06T 2207/10028; G06T 2207/10048; G06T 2207/30101; G06V 2201/03

(57) ABSTRACT

Cortical blood vessel temperature is automatically detected and tracked during an interventional procedure, such as during a neurosurgical procedure. In general, the temperature of blood vessels on the surface of the brain are identified and tracked using data from a brain thermography device, which may include an infrared (“IR”) thermal camera. Thermal data are segmented and vessel segments are identified and tracked based on the segmentation. The corresponding temperature measurements for each vessel segment are tracked over the frames of thermal data to track the temperature of blood vessels over time.

### STATEMENT OF FEDERALLY SPONSORED RESEARCH

This invention was made with government support under NS116190 and NS115362 awarded by the National Institutes of Health. The government has certain rights in the invention.

### BACKGROUND

The cortical blood vessels are one of the most prominent features in thermal images of the craniotomy. Under current understanding of how brain surface temperature changes over time, blood vessels act as the source of heat that is distributed across the surface, which eventually dissipates into the environment. Blood vessels are stable anatomical features which, through direct attachment to the cortical surface, experience motion in accordance with any linear or nonlinear deformations experienced by the rest of the cortical surface. In considering applications of infrared thermography to neurosurgery, measuring the temperature along the blood vessels over space and time is a potentially valuable preprocessing step.

### SUMMARY OF THE DISCLOSURE

The present disclosure addresses the aforementioned drawbacks by providing a method for detecting and tracking blood vessel temperature from thermal data acquired with a thermal imaging system. The method includes accessing thermal data with a computer system, where the thermal data include frames of thermal images acquired from a subject with a thermal imaging system and depicting at least one blood vessel of the subject. Segmented vessel data are generated from the thermal data using the computer system to segment the at least one blood vessel depicted in the thermal data. Vessel features are determined for each segmented blood vessel in the segmented vessel data using the computer system, and vessel segments are identified in the segmented vessel data based on the determined vessel features. A temperature along each vessel segment is measured by the computer system based on the thermal data. Each vessel segment is then tracked across the frames of thermal images using the computer system, and a report that indicates temperature measurements along at least one of the vessel segments as tracked across the frames of thermal images is then generated by the computer system.

It is another aspect of the present disclosure to provide a non-transitory computer-readable storage medium having stored thereon instruction that when executed by a processor cause the processor to receive thermal data by the processor, where the thermal data comprise frames of thermal images acquired from a subject with a thermal imaging system and depicting at least one blood vessel of the subject; generate segmented vessel data from the thermal data, the segmented vessel data including a plurality of vessel segments associated with the at least one blood vessel depicted in the thermal data; measure a temperature along each of the plurality of vessel segments based on the thermal data; track each of the plurality of vessel segments across the frames of thermal images; and output a report that indicates temperature measurements along at least one of the plurality of vessel segments as tracked across the frames of thermal images.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 is a flowchart illustrating the steps of an example method for the automated detection and tracking of cortical blood vessel temperature.

FIG. 2 illustrates an example of vessel temperature measurements and vessel path decomposition. Top left: The path of a single blood vessel measured in one frame of thermography video is shown. The green dot indicates the start point of the path, while the red dot indicates the end of the path. Selecting which side of the vessel is the starting point and which side of the vessel is the end point is arbitrary, but must remain consistent across the analysis. Top right: Temperature measured along the vessel path, from the start point to the end point. Bottom left: Horizontal component of the vessel trajectory. Bottom right: Vertical component of the vessel trajectory.

FIG. 3 is a flowchart illustrating the steps of an example method for segmenting vessels from thermal data, such as thermal images obtained with an infrared camera, or the like.

FIG. 4 illustrates an example process of vessel segmentation. Top left: A cropped frame of thermal data. Top center: Edge-enhancing effect of applying a Laplacian of Gaussian filter (sigma=9 pixels). Note that this step inverts the sign of peaks. Top right: Adaptive thresholding using a circular neighborhood with a radius of three sigma (27 pixels). Bottom left: Morphological thinning is performed until all structures are only one pixel in width. This process mirrors skeletonization but is computationally more efficient. Bottom center: Vessel endpoints and branching points are identified, and overlaid in red. Simple vascular segments are defined by the connecting region between endpoints and/or branching points. Bottom right: Vessels (blue) and endpoints (red) are overlaid over the raw data for visual inspection.

FIG. 5 illustrates an example of vessel segmentation algorithm performance by patient and sigma parameter. Segmentation results for three patients are shown. Left column: A cropped frame of raw thermal data from each patient. Right columns: Vessel segmentation outputs are shown for each patient, varying the standard deviation of the Laplacian of Gaussian filter from six to twelve pixels.

FIG. 6 illustrates example of vessel path errors. Different paths of a high-error vessel are shown. Each frame was specifically selected to demonstrate a specific form of vessel pathing error. Far left: The reference path (blue line) is shown for the vessel of interest, which branches from the center-left bifurcation down to the bottom-most end-point (red squares). Center left: This frame was selected to show high variability in vessel pathing without any changes to the fundamental structure. The proximal vessel branch point is slightly elevated compared to the regular path. The bend is smoother, and there is also less curvature towards the distal end point. Center right: This frame was selected as an example of path disconnection. In this case, the sampled vessel is significantly shorter than the reference path due to a missing proximal portion. Far right: The path is interrupted by an additional branch point in the bend, creating two simple vessel segments where there would typically just be one.

FIG. 7 illustrates an example process of vessel clustering. Full-frame vessel tracking was performed on thirty seconds of thermography video. Results were analyzed to assess analysis quality. Top Left: The spatial distribution of clustered vessels is shown. One colored circle is overlaid on the thermal frame for each vessel found during clustering. Colors were assigned to each circle at random to visualize possible overlaps. Each circle is centered at the vessel midpoint. The circle radius is proportional to the length of the vessel, and the opacity is equal to the percentage of the vessels found. Top right: The distribution of proportions at which each vessel was found is shown. Bottom left: The distribution of lengths for each vessel founds is shown. Bottom right: Found vessels were binned by length, and the average found proportion was calculated for each bin.

FIG. 8 illustrates example vessel thermograms. The temperature of a blood vessel is shown over thirty seconds. The vertical axis indicates distance along the blood vessel, while the horizontal axis indicates time in frame (camera frame rate=30 Hz). Top: The vessel thermogram as a direct output from vessel clustering is shown. Note the vertical striations introduced by variance in vessel path. Bottom: The vessel thermogram is shown after an alignment step. The vessel temperature in each frame was aligned to the vessel temperature in the previous frame using the function alignment algorithm in order to attenuate vertical striation artifacts.

FIG. 9 is a block diagram of an example system for automated detection and tracking of cortical blood vessel temperature.

FIG. 10 is a block diagram of example components that can implement the system of FIG. 9.

### DETAILED DESCRIPTION

Described here are systems and methods for the automated detection and tracking of cortical blood vessel temperature during an interventional procedure, such as during a neurosurgical procedure. In general, the systems and methods identify and track the temperature of blood vessels on the surface of the brain using data from a brain thermography device, which as a non-limiting example may include an infrared (“IR”) thermal camera. For instance, a brain thermography device such as those described in co-pending U.S. patent application Ser. No. 17/020,238, which is herein incorporated by reference in its entirety. Such thermography devices have various neurosurgical applications.

As one non-limiting example application, temperature can be measured and utilized as a metric of perfusion. An increase in local blood flow follows the functional activation of brain tissue, so measuring the change in temperature can be used to infer function. This technique can be used to monitor craniotomy blood flow during bypass surgery and provide early detection of cerebral hyperperfusion syndrome, among other conditions. As another non-limiting example application, the brain undergoes a nonlinear time-dependent deformation during neurosurgery known as brain shift. This is an unsolved problem in neurosurgery, and cortical blood vessels are stable anatomical features that could be used to perform surface-level brain registration.

The systems and methods described in the present disclosure can automatically detect and track cortical blood vessel temperature, which can be advantageous for the applications noted above. For instance, the systems and methods can operate in two modes: a “discovery mode,” in which all blood vessels within the field-of-view (“FOV”) are mapped; and a “real-time mode,” in which the temperature along a selected blood vessel can be tracked and measured in real-time. In the real-time mode, an average processing time on the order of 35 milliseconds per frame can be achieved. The speed and robustness of this technique are advantageous for its use in the neurosurgical applications described above.

### Real-Time Vessel Tracking

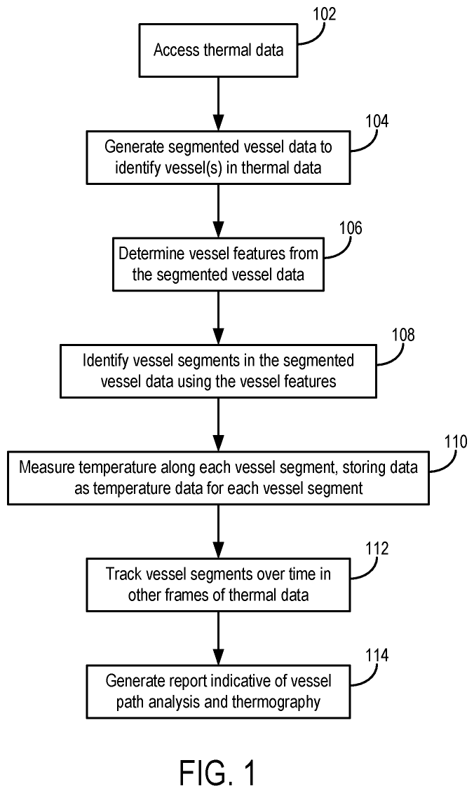

Referring now to FIG. 1, a flowchart is illustrated, showing the steps of an example method for temperature tracking blood vessels in real time using thermal data, such as thermal images acquired using a thermal imaging camera. The disclosed techniques enable a real-time intraoperative tool for temperature tracking of cortical blood vessels.

The method includes accessing thermal data with a computer system, as indicated at step 102. The thermal data can be accessed with the computer system by retrieving previously acquired thermal data from a memory or other data storage device or medium. Additionally or alternatively, the thermal data can be accessed with the computer system by acquiring the thermal data in real-time and transmitting or otherwise communicating the thermal data to the computer system. As described above, in some implementations the thermal data can include thermal imaging data, such as thermal imaging data acquired using an IR camera. In these instances, the thermal data can include images obtained with an IR camera. In some real-time implementations, thermal data can be loaded into memory from the thermal imaging system before the data are written to disk or another data storage device or medium.

As described below in more detail, the real-time algorithms described in the present disclosure generate vessel paths and temperatures for one vessel that has been selected manually, semi-automatically, or automatically, as opposed to generating thermograms for all vessels in an image. In some other implementations, more than one vessel may be selected for analysis. In practice, a consensus vessel skeleton is generated on a short interval of thermography or other thermal data, from which the target vessel can be chosen by the surgical team.

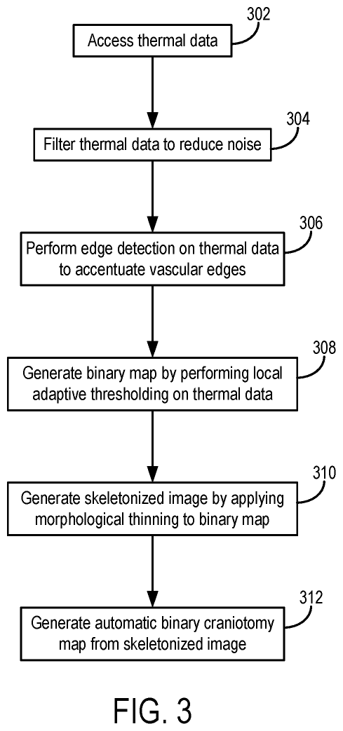

The method, therefore, includes identifying one or more vessels from the thermal data using the computer system, as indicated at step 104. Identifying one or more vessels in the thermal data can include performing a vessel segmentation, thereby generating segmented vessel data that includes segmented vessels for each frame of thermal data. A non-limiting example vessel segmentation technique is described in more detail below with respect to FIG. 3. For instance, a frame of thermal data can be segmented to generate a binary vessel mask, which may include a binary vascular skeleton. To reduce the computational burden of vessel segmentation, in some implementations the frame of thermal data can be cropped around the blood vessel to decrease the effective image size. The crop can be determined manually, semi-automatically, or automatically. For instance, the crop can be determined by determining the smallest possible box in which the vessel fits, then expanding the box to accommodate the filter sizes that will be used in the vessel segmentation process. As a non-limiting example, the filter sizes may be three Laplacian of Gaussian standard deviations in each cardinal directions (six standard deviations in total).

Vessel features are determined from the segmented vessel data, as indicated at step 106. For instance, the vessel features may include one or more of vessel segment end points, vessel segment bifurcation (or branch) points, vessel segment midpoints, vessel segment length, and so on.

The determined vessel features can be used to identify vessel segments in the segmented vessel data, as indicated at step 108. For instance, vessel segments can be determined as vessel paths extending between two end points, between an end point and a bifurcation point, between two bifurcation points, and the like. As will be described below, these vessel segments can be tracked over time in the thermal data to generate vessel thermograms.

In general, temperature measurements can be generated based on the automated segmentation of the vascular skeleton on a frame of thermal data. The vascular skeleton is therefore segmented into individual vascular segments, along which temperature can be measured. In a non-limiting example, the vascular skeleton is a binary mask of size equal to the input image, with pixels labeled as 1 denoting the midlines of major cortical blood vessels. The mask can be 8-connected, such that all pixels in all structures of area greater than one pixel have at least one adjacent pixel that is left, right, above, below, or any combination of two of these directions. The points can be further parsed in the vascular skeleton based on local connectivity.

Consider the 8-connected neighborhood of any individual pixel, and the number of non-connected pixels in this neighborhood. Points with zero neighbors are one-pixel islands, and can be ignored. Points with one neighbor, or one set of connected neighbors are end-points, which indicate the start or end of a blood vessel in the cortex. Points with three or more sets of non-connected neighbors are branch points, indicating where vessels overlap, converge, or bifurcate on the cortical surface. All other points can be defined as vessel points, and correspond to the body of cortical blood vessels. A vascular segment can be defined as an ordered set of pixels that connect two branch or end points. The choice of start and end point can be arbitrary. A simple vascular segment can be defined as a vascular segment that does not include any branch or end points.

This approach can be generalized to identify all blood vessels in a frame of thermal data. By applying the segmentation algorithm, the vascular skeleton can be obtained and therefore the set of all branch points and end points. Two branch or end points are considered neighbors if there exists a simple vascular segment that connects them. The set of all vascular segments may be obtained by iterating through all pairs of connected branch or end points. Two simple vascular segments are connected if they share a branch or end point. Let there be a vascular graph where each node is a simple vascular segment, and each edge is a branch or end point which connects two nodes. Then the set of all possible vessel paths is the set of all acyclic paths along that graph.

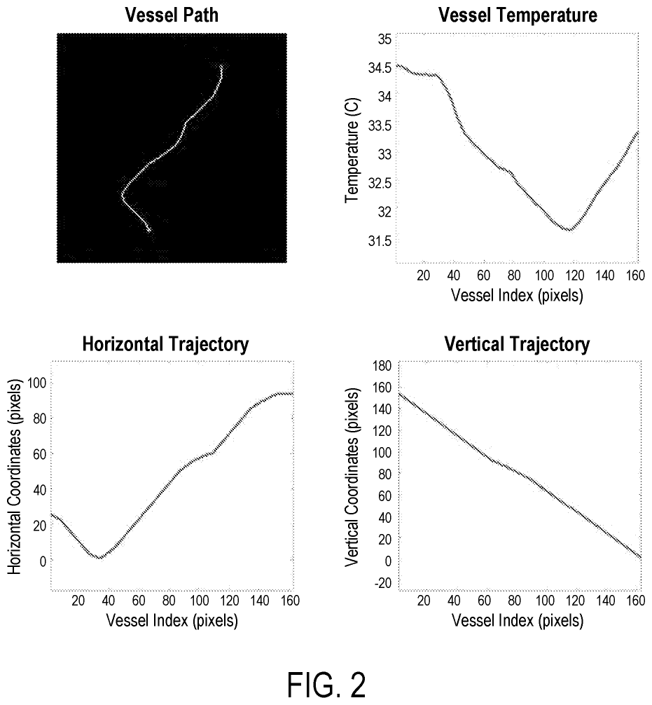

The temperature along each determined vessel path is then measured, as indicated at step 110, and stored as vessel segment temperature data. By evaluating the thermal image at the points in the vascular segment, the temperature can be measured along a vessel, as illustrated in FIG. 2.

The vessel segments identified in one frame of thermal data are then tracked over time in the other frames of thermal data, as indicated at step 112. As a non-limiting example, the vessel segments can be tracked using a clustering algorithm to match and track vessels and/or vessel segments across frames of thermal data.

As part of the vessel tracking process, rigid motion correction can be performed to move the initial vessel spatial parameters from the identified vessel(s) and/or vessel segment(s) onto the current thermal data frame. As a non-limiting example, the vascular bifurcations can be calculated from the vascular skeleton in the vessel segmentation data and used as features for point cloud registration. An example of a point cloud registration algorithm is described in more detail below. In general, the point cloud registration, or other motion correction technique, estimates a translation vector, matrix, or other transformation operator that can be used to move the initial vessel spatial parameters onto the current thermal data frame.

As another part of the vessel tracking process, vessel mapping can be performed. As an example, a mask can be constructed by dilating the skeleton from the vessel segmentation data of the previous thermal frame's blood vessel. As a non-limiting example, the skeleton can be dilated using a square element having a side length equal to the standard deviation of the Laplacian of Gaussian filter used in vessel segmentation. The constructed mask is then applied onto the existing thermal data frame. The current thermal data frame's blood vessel is then the shortest path along the vascular skeleton from the vessel start point to the vessel end point. The mask ensures that the new vessel is within the neighborhood of the previous thermal data frame's vessel, and slightly speeds up path estimation time.

By tracking one or more vessel segments over time, the corresponding temperatures measured at each frame of thermal data and associated with the tracked vessel segments can thus be monitored or otherwise analyzed, as indicated at step 114. For instance, a vessel thermogram can be generated based on the temperature measurements for one or more vessel segments as tracked over time.

A report is generated by the computer system based on the vessel path analysis, as indicated at step 116. As one example, the report can include textual information, images, and/or other data that may be compiled and displayed or otherwise presented to a user. For instance, the report may include thermograms and/or other data output from the vessel path analysis and displayed to a user via a graphical user interface of the computer system during a neurosurgical procedure. In this way, the report can provide the user (e.g., a neurosurgeon) with data about the vessel temperature tracking in real-time while the neurosurgical procedure is being performed.

### Vessel Segmentation

As described above, one step of the method for automated detection and tracking of cortical blood vessel temperature during an interventional procedure is identifying vessels in thermal data. As a non-limiting example, vessels can be identified in thermal data using a vessel segmentation technique. To facilitate segmenting vessels from a single frame of thermal data, blood vessels can be assumed to have a number of properties. For example, the blood vessels are warmer than the surrounding cortex. While the physical edges of a blood vessel are sharp, in a thermal image the vessel edges are blurred by diffusion. Nonetheless, the spatial temperature gradients in the vicinity of the edge of a blood vessel are larger than those in ordinary cortex. The major blood vessels are relatively sparsely distributed, with smaller branches extending into the nearby tissue, and occasional vascular intersections, or branching points. Additionally, all the blood vessels are on the brain surface, which is interior to the craniotomy edge. From these properties a general approach for blood vessel segmentation can be established.

Referring now to FIG. 3, a flowchart is illustrated, showing the steps of an example method for generating vessel segmentation data from thermal data, where the vessel segmentation data represent segmented blood vessels.

Thermal data are accessed with a computer system, as indicated at step 302. The thermal data can be accessed with the computer system by retrieving previously acquired thermal data from a memory or other data storage device or medium. Additionally or alternatively, the thermal data can be accessed with the computer system by acquiring the thermal data in real-time and transmitting or otherwise communicating the thermal data to the computer system. As described above, in some implementations the thermal data can include thermal imaging data, such as thermal imaging data acquired using an IR camera. In these instances, the thermal data can include images obtained with an IR camera.

The thermal data are filtered using the computer system to reduce noise, as indicated at step 304. As a non-limiting example, a Gaussian smoothing filter can be applied to the thermal data in order to attenuate any speckle noise.

Edge detection is performed on the thermal data using the computer system, as indicated at step 306. As a non-limiting example, a Laplacian filter can be applied to the thermal data using the computer system, in order to accentuate the vascular edges.

A binary image is generated from the thermal data using the computer system, as indicated at step 308. As a non-limiting example, the binary map can be generated by applying a local adaptive threshold, where each pixel is set to unity if their value exceeds the average of nearby pixels, and set to zero otherwise.

A skeletonized image is generated from the binary image using the computer system, as indicated at step 310. As a non-limiting example, repeated morphological thinning is applied to the binary image until only a one-pixel-wide skeleton remains.

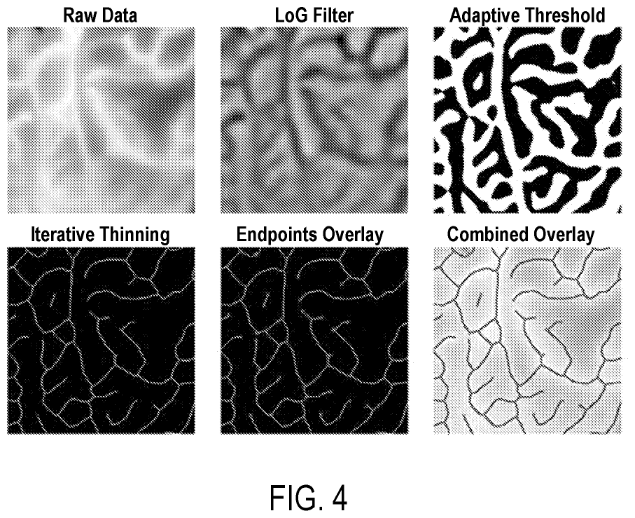

An automatic binary craniotomy mask is then generated from the skeletonized image using the computer system, as indicated at step 312, which can then applied to the image to remove any skeleton pixels from the surgical site periphery. This process is visualized in FIG. 4, which illustrates a cropped frame of thermal data (top left), edge-enhancing effect of applying a Laplacian of Gaussian (“LoG”) filter (sigma=9 pixels) to the thermal data (top center). Note that this step inverts the sign of peaks. Adaptive thresholding using a circular neighborhood with a radius of three sigma (27 pixels) is applied to the thermal data (top right). Morphological thinning is performed until all structures are only one pixel in width (bottom left). This process mirrors skeletonization, but is computationally more efficient. Vessel endpoints and branching points are identified, and overlaid in red (bottom center). Simple vascular segments are defined by the connecting region between endpoints and/or branching points. Vessels (blue) and endpoints (red) are overlaid over the raw data for visual inspection (bottom right).

Advantageously, the vessel segmentation technique described above with respect to FIG. 3 can be readily adaptable to different patient cases, with different camera positions and orientations. Of the filters described above, two require individual parameter setting: the Gaussian smoothing filter, and the adaptive threshold filter. To set these parameters well, the role of each filter within the scope of the processing pipeline can be considered. The Gaussian smoothing filter attenuates spatial patterns that are smaller than individual blood vessels, and sets up the Laplacian to be smooth. As a sliding summative metric of the second derivative, the Laplacian filter can be interpreted as a measure of local concavity or convexity. The inflection point on a Gaussian curve, where the Laplacian evaluates to zero, is one standard deviation away from the mean. Therefore, the Laplacian of a Gaussian filter can accentuate objects that are comparable in size to double the Gaussian standard deviation. As cortical blood vessels are typically about 1 mm in width, this value can be used as the Gaussian standard deviation. In other instances, different values can be used as the Gaussian standard deviation where different vessel diameters are expected. The width of the Laplacian of Gaussian filter can then be set to five times the standard deviation in either direction, for a total of 1 cm in the example noted above. This parameter can be tuned to trade off precision for processing speed.

The shape of the adaptive threshold filter specifies a local neighborhood around each vessel through a circular disk element. For the vessel to be comfortably distinguished from the surrounding area, the radius of the filter can be set to be at least equal to twice the radius of the Gaussian filter. This ensures that the vessel pixels are a minority within each neighborhood, and will have Laplacian values of higher magnitude than the average Laplacian value in that neighborhood. For robustness, the radius of the adaptive filter can be set to three times the standard deviation of the Gaussian filter, or about 3 mm in the example noted above.

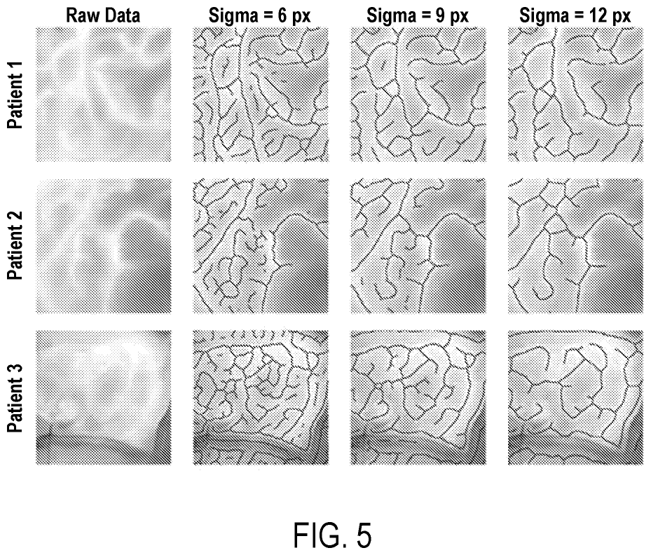

In an example study, the vessel segmentation algorithm performance by sigma value is compared by patient in FIG. 5. Overall, the algorithm is able to identify vessels across patient cases, even when sigma is changed by up to 50%. As sigma is increased, the space between vessels also increases, so sigma can be interpreted as an area filter parameter. Note that the major vessels were segmented regardless of sigma value, with most of the difference between images being in the density and accuracy of smaller vessels. While in this example study 9 pixels performed well for most cases overall, the sigma value can be readily optimized for a specific patient, as described above. In practice, the sigma value can be manually or semi-automatically set using a user interface. For example, a user (e.g., a neurosurgeon) can be presented with a graphical user interface in which a sliding bar is displayed as a control element for controlling the sigma value. The optimal value of sigma is related to the edge-length of a single pixel. For example, the image of Patient 3 in FIG. 5 was collected farther away from Patient 1 and Patient 2, and so a lower sigma value performs better as the edge-length per pixel is larger as well.

### Vessel Tracking

A technique for segmenting and measuring temperature along blood vessels in a single frame is described above; however, practical applications of thermography typically measure changes in temperature over time, and therefore the segmentation of the same vessel or set of vessels is tracked across a thermal video, as noted above. Because videos are composed of many sequential frames, the same vessel segmentation technique may be applied on each frame. While this is a reasonable first step, several limitations may be encountered. Thus, in some implementations a vessel tracking algorithm can be implemented to more robustly track vessel segments across frames of thermal data.

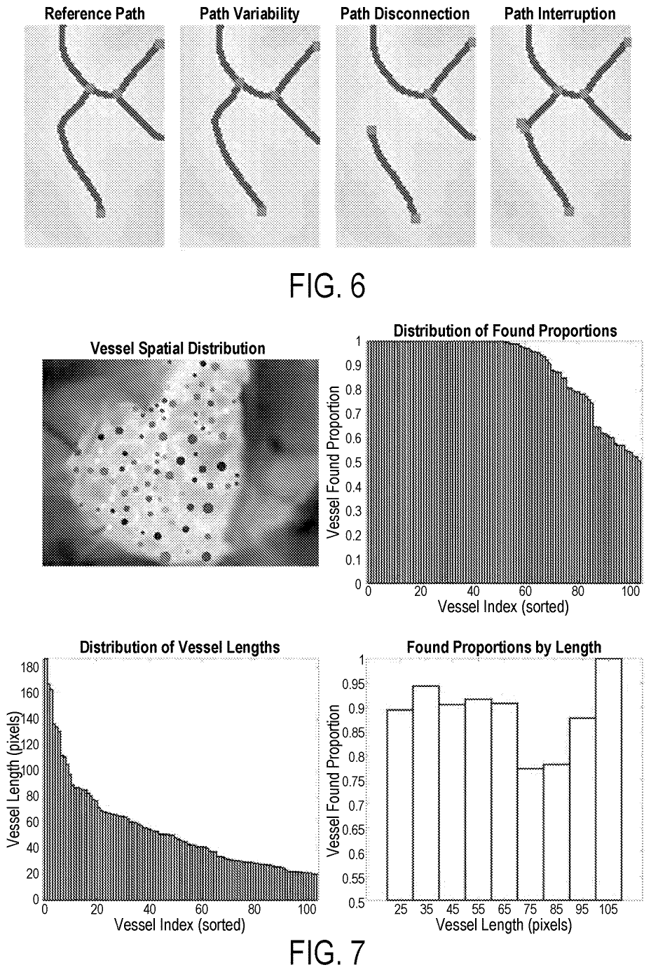

In some instances, the same vessel may not appear in every frame. This may be more common in blurrier parts of an image, or areas that are otherwise diffusely warm. The precise vessel morphology is clouded, and higher variance in vessel shapes is observed. Frequently, two nearby vessels may merge or split between frames. This occurs when both vessel segments are defined by a branch point and an end point on the vascular skeleton, resulting in one merged vessel, which may be marked by two branch points (see FIG. 6 for visual examples of this and other vessel path errors). It is also expected that there may be small fluctuations in the vessel path. In order to robustly estimate the existence of a blood vessel, some consensus must be reached regarding the location and shape of the vessels present in the image.

A set of robust vessel features can be developed and used to identify the same vessel in different images. Vessels can then be matched from different frames by performing clustering in the vessel feature space. First, it can be assumed that the amount of motion of a blood vessel across a video is small enough to be ignored over the short periods of time in which vessel tracking is performed. Although the brain undergoes motion due to various sources, much of the high-amplitude motion can be attenuated using the point cloud registration method discussed later in this section. By calculating the frame-to-frame transformation for each pair of subsequent frames and applying the inverse transformation, any motion described by a linear transformation can be removed, leaving only nonrigid motion sources, which are typically lower in amplitude and frequency. Second, it can be assumed that the vessel path is relatively consistent across frames. This can be assumed because the vessel corresponds to an anatomical structure which exists in each frame. Any vessel with a large path variance due to processing errors likely cannot be measured in any case.

A consequence of these two assumptions is that the vessel midpoint will occupy a similar position across frames. Similarly, the vessel will have a similar length across all frames. A simple vessel “fingerprint” can then be made by concatenating the vessel length with the vessel midpoint. Two different simple vessel segments cannot have the same midpoint. The Laplacian of Gaussian filter used above nearly guarantees that the body of each vessel will be far away from the body of any other vessel, except near intersection points. However, the midpoint is by definition the point along the vessel body that is furthest from any intersection points. Meanwhile, the vessel length can be helpful when there is high variability in estimated vascular anatomy due to merging and splitting of nearby vessels.

These features have been found to be sufficient in the cases tested. A general issue with designing vessel path features is finding properties which are invariant to vessel length. Variance in vessel paths appears to be larger in smaller vessels, which can be amplified if the features are of higher complexity. A second challenge is finding features that are likely unique for each vessel. A third challenge is scaling the feature dimensions such that the distance metric preserves spatial meaning. For these features, the units are in pixels and Euclidean distance may be conveniently used.

Using a set of blood vessels for each frame in thermal data, each vessel and/or vessel segment can be assigned a point in the vessel fingerprint space. It is expected that different instances of the same blood vessel segment across the frames will inhabit a similar region in the fingerprint space. The vessel matching problem can thus be redefined as a clustering problem. One property of the clustering problem is that the number of clusters is not known, as it is unclear how many blood vessel segments are in the image prior to clustering. Another property of the clustering problem is that clusters are easily separable using Euclidean distance, as due to the effects of the Laplacian of Gaussian filter it can be expected for vessels to be at least one Gaussian standard deviation away from one another. These properties lend themselves to a hierarchical clustering approach, using the Gaussian standard deviation as the cutoff distance between clusters.

The effectiveness of hierarchical clustering in identifying vessel clusters in an example study is shown in FIG. 7. The spatial distribution of blood vessels is uniform throughout the image, and there is no intersection of circles indicating wide spacing between blood vessel midpoints. High overall opacity indicates vessels are frequently found on each frame, and there is no clear spatial distribution for length or found frequency variables. Furthermore, most of the vessels were found in every frame. No vessels were found less than half of the time, which is provided by the cluster filtering approach. Found vessel lengths ranged from 20 pixels minimum up to over 180 pixels long. Lastly, found vessel proportions had no clear monotonic relationship to vessel length.

The next step in the vessel tracking process is to filter and process the vessel clusters. Each vessel cluster can be interpreted as a structural pattern detected in the vascular skeleton across time. When there is no filtering step prior to clustering, noisy or errorful vessel segments will be assigned clusters alongside true vessel segments. The goal of filtering is to measure a consensus of which vessel structures are consistently found across time. Consistency can be defined as the presence of the blood vessel in a majority of video frames, filtering out any cluster with size less than or equal to half the number of video frames. This enforces that, where multiple variations of the blood vessel exist, only one of those iterations is chosen and vessels are not duplicated even in extreme conditions. While this approach may exclude some vessels without a majority (e.g., a plurality case), these vessels are likely too noisy to be measured reliably.

Subsequent filters can be applied to ensure additional cluster properties. The same vessel cannot be found in more instances than the total number of frames in a video, so any clusters of size larger than the number of frames may also be filtered out. Although not expected, this can occur if the filtering cutoff is set too high. Each cluster should have at most one instance of a blood vessel segment from each individual frame; the set of frame indices corresponding to the vessels in a cluster must be unique. It may be practical for some analyses to set a lower bound on the vessel length. In these cases the median length of all found vessels in each cluster may be calculated and compared to a cutoff length supplied by the user.

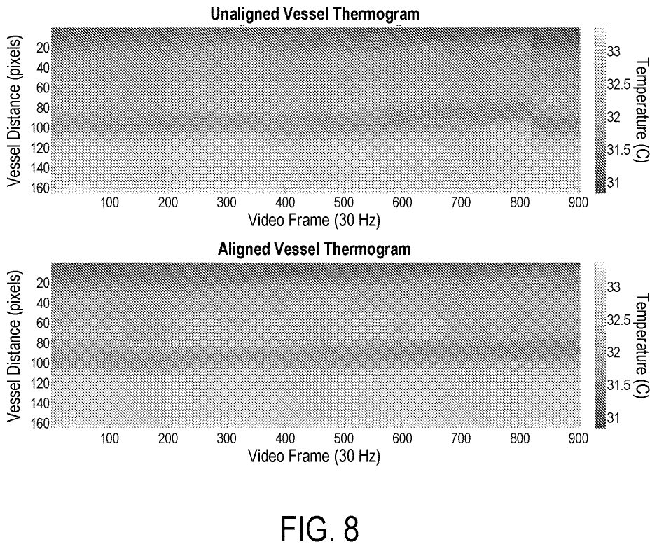

With the filtered set of blood vessels defined by hierarchical clusters, each cluster is a set of matching blood vessel segments. Each blood vessel segment is an ordered list of pixels, containing two spatial component coordinates and the corresponding temperature. This information can be aggregated into a measurement of the vessel temperature over time. For each vessel, a number of points to sample on each frame can be picked, such as the median number of pixels across all frames where the vessel is found. Each sampled point corresponds to a position along the vessel. A vessel thermogram can be defined as a data matrix where one dimension samples the vessel temperature over space, and the other dimension samples the vessel temperature over time. Constructing a thermogram for each vessel allows easy visualization of temperature changes. Furthermore, it permits quantitative study of the vessel across space and time by ensuring the input data to the analysis is of rectangular shape. An example thermogram is shown in FIG. 8.

Vessel data has two types of problems that may inhibit direct measurement of the thermogram. First, the vessel may not be found on every frame (missing data problem). Second, the vessel may change in length across frames (noisy data problem). To address the missing data problem, the temperatures for missing frames can be interpolated from neighboring frames. For an approach that is real-time compatible, the vessel temperature from the previous frame may be used. The noisy data problem can be addressed with a more sophisticated approach. Blood vessels do not experience large fluctuations in length on a time scale similar to the image sampling rate. Differences in the paths between adjacent frames are more likely due to changes in vessel start and end points, or small changes in the vessel path. This can be framed as a global sequence alignment problem in one dimension.

### Point Cloud Registration Algorithm

A fast, between-frame vessel alignment technique implemented as a point cloud registration algorithm can be used to address the global sequence alignment problem noted above. Let there be two point clouds A and B with N and M points respectively, which sample features of an object sampled at two different times. Suppose that the process for sampling points is noisy, introducing errors drawn from a known distribution, ε, and that the sampling process is random, where each point is sampled without replacement with an unknown probability, p.

The object undergoes a transformation T(x|θ) during this interval, such that T(Aᵢ)∈B for any point Aᵢ∈A which has a matching point in B, and θ is the set of parameters that define the transformation T. It is not known which points match, only that some of the points do match, and the sets corresponding to the point clouds A and B are in no particular order. The objective is to solve for the transformation function T(x) given the point clouds A, B, and the distribution ε. As a non-limiting example, maximizing the conditional likelihood of observing B given the transformation parameters θ, and using the maximum likelihood parameters can be done to establish T:

$$f ( x \vert θ ) = {\sum }_{i = 1}^{N} T ( δ ( x - {A}_{i} ) \vert θ ) * ε$$

$$g ( θ ) = {\sum }_{j = 1}^{M} f ( {B}_{j} \vert θ )$$

$$\hat{θ} = arg max g ( θ )$$

where δ is the Dirac delta function, \hat{θ} is the estimated transformation parameters, and * indicates convolution. This approach has a couple of advantages for real-world applications compared to other point registration methods. First, it is designed to handle mismatches between point clouds. For example, if a point in B has no nearby point in A, then f and its gradient with respect θ will both be small relative to points in B with nearby points in A, as it is distant from any convolution with ε. Alternatively, if a point in A has no nearby points in B, then f will not be evaluated near A, and this point will have no strong contribution to g(θ). Second, this method does not need an equal number of points in A and B, nor does it require a known relationship between points in A and B. Further, the cost function g(θ) is composed mostly of function evaluations and summations, which makes it easy to differentiate and optimize θ depending on the transformation complexity and choice of ε.

This point cloud registration approach can be applied to vessel tracking as follows. Let A be a two-dimensional point cloud corresponding to vascular bifurcations in a thermal image, and let B be the vascular bifurcations in the subsequent frame. It can be assumed that the sampling rate of the camera is of higher frequency than the frequencies corresponding to rigid motion. This implies that the magnitude of the translation vector between frames is small enough to be tracked over time. Furthermore, the minimum distance between bifurcations is closely related to the standard deviation of the Laplacian of Gaussian filter used in vessel segmentation. The magnitude of the motion between frames is lower than this standard deviation. Therefore, the local maximum of g(θ) is near or equivalent to the global maximum. Second, the segmentation algorithm is stable over time. Suppose that the motion between frames can be described by a translation, and that the error with which bifurcations are sampled is normally distributed.

$$ε \sim N ( 0 , σ )$$

$$θ = ( s , t )$$

$$T ( ( x , y ) \vert θ ) = ( x + s , y + t )$$

$$f ( x \vert θ ) = {\sum }_{i = 1}^{N} N ( ( {A}_{i x} + s , {A}_{i y} + t ) , σ )$$

The resulting cost function is the summed evaluations of a function, which is the sum of many Gaussians. This function is smooth and easily differentiable, which allows maximizing g(θ) through gradient descent. This application of the point registration algorithm ensures that adjacent frames are rigidly aligned, which is advantageous for generating vessel thermograms.

### Computer Systems

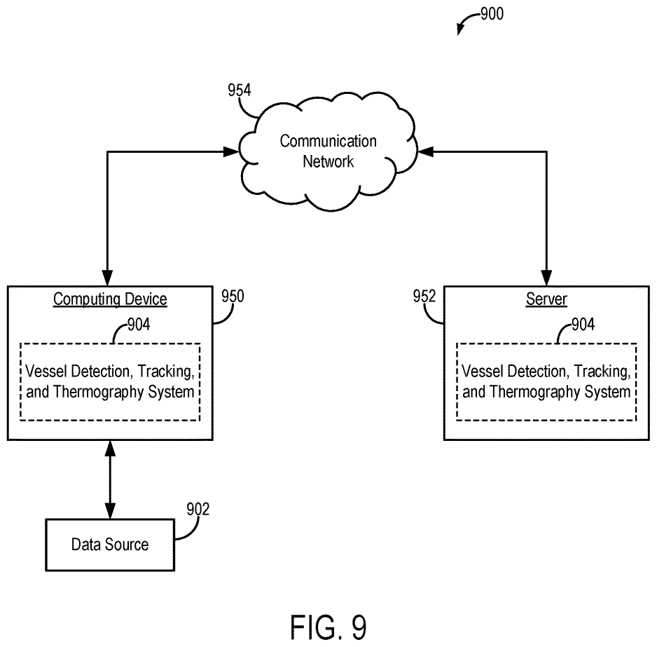

Referring now to FIG. 9, an example of a system 900 for automated detection and tracking of cortical blood vessel temperature in accordance with some embodiments of the systems and methods described in the present disclosure is shown. As shown in FIG. 9, a computing device 950 can receive one or more types of data (e.g., thermal data) from data source 902. In some embodiments, computing device 950 can execute at least a portion of a vessel detection, tracking, and thermography system 904 to detect and track blood vessel temperature from thermal data received from the data source 902.

Additionally or alternatively, in some embodiments, the computing device 950 can communicate information about data received from the data source 902 to a server 952 over a communication network 954, which can execute at least a portion of the vessel detection, tracking, and thermography system 904. In such embodiments, the server 952 can return information to the computing device 950 (and/or any other suitable computing device) indicative of an output of the vessel detection, tracking, and thermography system 904.

In some embodiments, computing device 950 and/or server 952 can be any suitable computing device or combination of devices, such as a desktop computer, a laptop computer, a smartphone, a tablet computer, a wearable computer, a server computer, a virtual machine being executed by a physical computing device, and so on. The computing device 950 and/or server 952 can also reconstruct images from the data.

In some embodiments, data source 902 can be any suitable source of data (e.g., thermal measurement data, thermal images reconstructed from measurement data, processed thermal image data), such as a thermal imaging system, another computing device (e.g., a server storing thermal measurement data, images reconstructed from thermal measurement data, processed thermal image data), and so on. In some embodiments, data source 902 can be local to computing device 950. For example, data source 902 can be incorporated with computing device 950 (e.g., computing device 950 can be configured as part of a device for measuring, recording, estimating, acquiring, or otherwise collecting or storing data). As another example, data source 902 can be connected to computing device 950 by a cable, a direct wireless link, and so on. Additionally or alternatively, in some embodiments, data source 902 can be located locally and/or remotely from computing device 950, and can communicate data to computing device 950 (and/or server 952) via a communication network (e.g., communication network 954).

In some embodiments, communication network 954 can be any suitable communication network or combination of communication networks. For example, communication network 954 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 954 can be a local area network, a wide area network, a public network (e.g., the Internet), a private or semi-private network (e.g., a corporate or university intranet), any other suitable type of network, or any suitable combination of networks. Communications links shown in FIG. 9 can each be any suitable communications link or combination of communications links, such as wired links, fiber optic links, Wi-Fi links, Bluetooth links, cellular links, and so on.

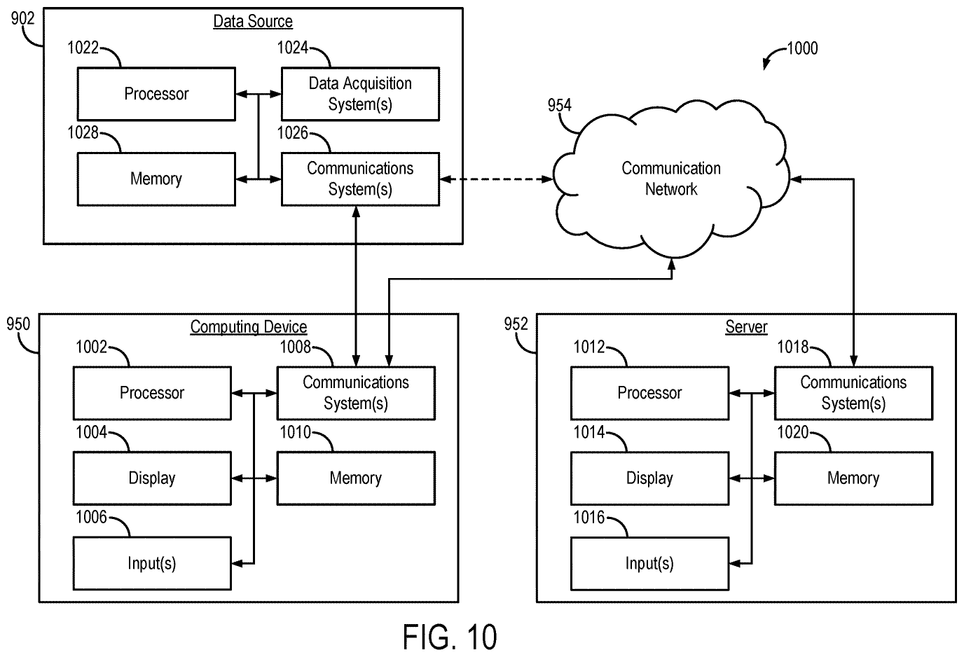

Referring now to FIG. 10, an example of hardware 1000 that can be used to implement data source 902, computing device 950, and server 952 in accordance with some embodiments of the systems and methods described in the present disclosure is shown.

As shown in FIG. 10, in some embodiments, computing device 950 can include a processor 1002, a display 1004, one or more inputs 1006, one or more communication systems 1008, and/or memory 1010. In some embodiments, processor 1002 can be any suitable hardware processor or combination of processors, such as a central processing unit (“CPU”), a graphics processing unit (“GPU”), and so on. In some embodiments, display 1004 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1006 can include any suitable input devices and/or sensors that can be used to receive user input, such as a keyboard, a mouse, a touchscreen, a microphone, and so on.

In some embodiments, communications systems 1008 can include any suitable hardware, firmware, and/or software for communicating information over communication network 954 and/or any other suitable communication networks. For example, communications systems 1008 can include one or more transceivers, one or more communication chips and/or chip sets, and so on. In a more particular example, communications systems 1008 can include hardware, firmware, and/or software that can be used to establish a Wi-Fi connection, a Bluetooth connection, a cellular connection, an Ethernet connection, and so on.

In some embodiments, memory 1010 can include any suitable storage device or devices that can be used to store instructions, values, data, or the like, that can be used, for example, by processor 1002 to present content using display 1004, to communicate with server 952 via communications system(s) 1008, and so on. Memory 1010 can include any suitable volatile memory, non-volatile memory, storage, or any suitable combination thereof. For example, memory 1010 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1010 can have encoded thereon, or otherwise stored therein, a computer program for controlling operation of computing device 950. In such embodiments, processor 1002 can execute at least a portion of the computer program to present content (e.g., images, user interfaces, graphics, tables), receive content from server 952, transmit information to server 952, and so on. For example, the processor 1002 and the memory 1010 can be configured to perform the methods described herein (e.g., the method of FIG. 1; the method of FIG. 3; the vessel tracking methods described above; the point cloud registration methods described above; and so on).

In some embodiments, server 952 can include a processor 1012, a display 1014, one or more inputs 1016, one or more communications systems 1018, and/or memory 1020. In some embodiments, processor 1012 can be any suitable hardware processor or combination of processors, such as a CPU, a GPU, and so on. In some embodiments, display 1014 can include any suitable display devices, such as an LCD screen, LED display, OLED display, electrophoretic display, a computer monitor, a touchscreen, a television, and so on. In some embodiments, inputs 1016 can include any suitable input devices and/or sensors that can be used to receive user input, such as a keyboard, a mouse, a touchscreen, a microphone, and so on.

In some embodiments, communications systems 1018 can include any suitable hardware, firmware, and/or software for communicating information over communication network 954 and/or any other suitable communication networks. For example, communications systems 1018 can include one or more transceivers, one or more communication chips and/or chip sets, and so on. In a more particular example, communications systems 1018 can include hardware, firmware, and/or software that can be used to establish a Wi-Fi connection, a Bluetooth connection, a cellular connection, an Ethernet connection, and so on.

In some embodiments, memory 1020 can include any suitable storage device or devices that can be used to store instructions, values, data, or the like, that can be used, for example, by processor 1012 to present content using display 1014, to communicate with one or more computing devices 950, and so on. Memory 1020 can include any suitable volatile memory, non-volatile memory, storage, or any suitable combination thereof. For example, memory 1020 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1020 can have encoded thereon a server program for controlling operation of server 952. In such embodiments, processor 1012 can execute at least a portion of the server program to transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone), and so on.

In some embodiments, the server 952 is configured to perform the methods described in the present disclosure. For example, the processor 1012 and memory 1020 can be configured to perform the methods described herein (e.g., the method of FIG. 1; the method of FIG. 3; the vessel tracking methods described above; the point cloud registration methods described above; and so on).

In some embodiments, data source 902 can include a processor 1022, one or more data acquisition systems 1024, one or more communications systems 1026, and/or memory 1028. In some embodiments, processor 1022 can be any suitable hardware processor or combination of processors, such as a CPU, a GPU, and so on. In some embodiments, the one or more data acquisition systems 1024 are generally configured to acquire data, images, or both, and can include a thermal imaging system. Additionally or alternatively, in some embodiments, the one or more data acquisition systems 1024 can include any suitable hardware, firmware, and/or software for coupling to and/or controlling operations of a thermal imaging system. In some embodiments, one or more portions of the data acquisition system(s) 1024 can be removable and/or replaceable.

Note that, although not shown, data source 902 can include any suitable inputs and/or outputs. For example, data source 902 can include input devices and/or sensors that can be used to receive user input, such as a keyboard, a mouse, a touchscreen, a microphone, a trackpad, a trackball, and so on. As another example, data source 902 can include any suitable display devices, such as an LCD screen, an LED display, an OLED display, an electrophoretic display, a computer monitor, a touchscreen, a television, etc., one or more speakers, and so on.

In some embodiments, communications systems 1026 can include any suitable hardware, firmware, and/or software for communicating information to computing device 950 (and, in some embodiments, over communication network 954 and/or any other suitable communication networks). For example, communications systems 1026 can include one or more transceivers, one or more communication chips and/or chip sets, and so on. In a more particular example, communications systems 1026 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1028 can include any suitable storage device or devices that can be used to store instructions, values, data, or the like, that can be used, for example, by processor 1022 to control the one or more data acquisition systems 1024, and/or receive data from the one or more data acquisition systems 1024; to generate images from data; present content (e.g., data, images, a user interface) using a display; communicate with one or more computing devices 950; and so on. Memory 1028 can include any suitable volatile memory, non-volatile memory, storage, or any suitable combination thereof. For example, memory 1028 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1028 can have encoded thereon, or otherwise stored therein, a program for controlling operation of data source 902. In such embodiments, processor 1022 can execute at least a portion of the program to generate images, transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for detecting and tracking blood vessel temperature from thermal data acquired with a thermal imaging system, the method comprising:
(a) accessing thermal data with a computer system, wherein the thermal data comprise frames of thermal images acquired from a subject with a thermal imaging system and depicting at least one blood vessel of the subject;
(b) generating segmented vessel data from the thermal data using the computer system to segment the at least one blood vessel depicted in the thermal data;
(c) determining vessel features for each segmented blood vessel in the segmented vessel data using the computer system;
(d) identifying, by the computer system, vessel segments in the segmented vessel data based on the determined vessel features;
(e) measuring, by the computer system, a temperature along each vessel segment based on the thermal data;
(f) tracking each vessel segment across the frames of thermal images using the computer system; and
(g) generating a report that indicates temperature measurements along at least one of the vessel segments as tracked across the frames of thermal images.

2. The method of claim 1, wherein segmenting the thermal data to generate the segmented vessel data comprises generating a binary map for each frame of the thermal data.

3. The method of claim 2, wherein generating the segmented vessel data further comprises generating a vascular skeleton map for each frame of thermal data by skeletonizing each binary map.

4. The method of claim 1, where determined vessel features comprise at least one of vessel segment end points, vessel segment branch points, vessel segment midpoints, or vessel segment lengths.

5. The method of claim 4, wherein the determined vessel features comprise vessel segment end points and vessel segment branch points.

6. The method of claim 4, wherein vessel segments are identified based on vessel paths extending from at least one of:
a first vessel segment end point to a second vessel segment end point;
a vessel segment end point to a vessel segment branch point; or
a first vessel segment branch point to a second vessel segment branch point.

7. The method of claim 1, wherein tracking each vessel segment across the frames of thermal images in the thermal data comprises:
generating vessel segment feature data by determining vessel segment features for each vessel segment;
generating clustered vessel segment feature data by applying a clustering algorithm to the vessel segment feature data; and
tracking each vessel segment based on the clustered vessel segment feature data.

8. The method of claim 7, wherein the clustering algorithm comprises a hierarchical clustering algorithm.

9. The method of claim 7, wherein the vessel segment features comprise a vessel segment midpoint and a vessel segment length.

10. The method of claim 1, wherein tracking each vessel segment across the frames of thermal images in the thermal data further comprises applying a point cloud registration to vessel features in each frame of thermal data.

11. The method of claim 1, wherein generating the report includes generating a thermogram for a vessel segment based on temperature measurements for that vessel segment as tracked in each frame of the thermal data.

12. The method of claim 11, wherein the thermogram comprises a data matrix having a first dimension that samples the vessel segment temperature measurement over space and a second dimensions that samples the vessel segment temperature measurement over time.

13. A non-transitory computer-readable storage medium having stored thereon instruction that when executed by a processor cause the processor to perform a method comprising:
receiving thermal data by the processor, wherein the thermal data comprise frames of thermal images acquired from a subject with a thermal imaging system and depicting at least one blood vessel of the subject;
generating segmented vessel data from the thermal data, the segmented vessel data comprising a plurality of vessel segments associated with the at least one blood vessel depicted in the thermal data;
measuring a temperature along each of the plurality of vessel segments based on the thermal data;
tracking each of the plurality of vessel segments across the frames of thermal images; and
outputting a report that indicates temperature measurements along at least one of the plurality of vessel segments as tracked across the frames of thermal images.

14. The non-transitory computer readable storage medium of claim 13, wherein generating the segmented vessel data comprises generating a binary map for each frame of the thermal data.

15. The non-transitory computer readable storage medium of claim 14, wherein generating the segmented vessel data further comprises generating a vascular skeleton map for each frame of thermal data by skeletonizing each binary map.

16. The non-transitory computer readable storage medium of claim 13, wherein tracking each vessel segment across the frames of thermal images in the thermal data comprises:
generating vessel segment feature data by determining vessel segment features for each vessel segment;
generating clustered vessel segment feature data by applying a clustering algorithm to the vessel segment feature data; and
tracking each vessel segment based on the clustered vessel segment feature data.

17. The non-transitory computer readable storage medium of claim 16, wherein the clustering algorithm comprises a hierarchical clustering algorithm.

18. The non-transitory computer readable storage medium of claim 16, wherein the vessel segment features comprise a vessel segment midpoint and a vessel segment length.

19. The non-transitory computer readable storage medium of claim 13, wherein tracking each vessel segment across the frames of thermal images in the thermal data comprises applying a point cloud registration to vessel features in each frame of thermal data.

20. The non-transitory computer readable storage medium of claim 13, wherein outputting the report comprises generating a thermogram for one of the plurality of vessel segments based on temperature measurements for that vessel segment as tracked in each frame of the thermal data.
